(19) United States
(12) Patent Application Publication
(10) Pub. No.: US 2025/0281089 A1
(43) Pub. Date: Sep. 11, 2025

(54) METHOD FOR PROVIDING BIOMETRIC INFORMATION AND ELECTRONIC DEVICE SUPPORTING SAME

(71) Applicant: Samsung Electronics Co., Ltd., Suwon-si (KR)

(72) Inventors: Donghyun LEE, Suwon-si (KR); Intaek OH, Suwon-si (KR)

(21) Appl. No.: 19/215,798

(22) Filed: May 22, 2025

Related U.S. Application Data
(63) Continuation of application No. PCT/KR2023/019887, filed on Dec. 5, 2023.

(30) Foreign Application Priority Data
Dec. 5, 2022 (KR) 10-2022-0167841
Jan. 4, 2023 (KR) 10-2023-0000990

(51) Int. Cl. A61B 5/16; A61B 5/00; A61B 5/024; A61B 5/11; A61B 5/18; B60W 40/08; B60W 50/08; B60W 50/14

(52) U.S. Cl. CPC A61B 5/165; A61B 5/02416; A61B 5/1112; A61B 5/18; A61B 5/4809; A61B 5/7264; A61B 5/7405; A61B 5/742; A61B 5/7475; B60W 40/08; B60W 50/082; B60W 50/14; A61B 2503/22; A61B 2560/0462; A61B 2562/0219; B60W 2040/0827; B60W 2050/146

(57) ABSTRACT

A wearable device and an electronic device included in a vehicle are provided. The wearable device includes at least one sensor, a communication interface, at least one processor, and memory, comprising one or more storage media, storing instructions, wherein the instructions, when executed by the at least one processor individually or collectively, cause the wearable device to obtain first data used to calculate a user's biometric level by using the at least one sensor, transmit the first data to an external electronic device, receive guide information related to a change in at least part of the biometric level from the external electronic device, based on the transmission of the first data, obtain second data related to properties of the guide information by using the at least one sensor, based on the reception of the guide information, and transmitting the second data to the external electronic device.

### CROSS-REFERENCE TO RELATED APPLICATION(S)

This application is a continuation application, claiming priority under 35 U.S.C. § 365(c), of an International application No. PCT/KR2023/019887, filed on Dec. 5, 2023, which is based on and claims the benefit of a Korean patent application number 10-2022-0167841, filed on Dec. 5, 2022, in the Korean Intellectual Property Office, and of a Korean patent application number 10-2023-0000990, filed on Jan. 4, 2023, in the Korean Intellectual Property Office, the disclosure of each of which is incorporated by reference herein in its entirety.

### BACKGROUND

### 1. Field

The disclosure relates to a method for providing biometric information and an electronic device supporting the same.

### 2. Description of Related Art

With the advancement of digital convergence in which various information and communication technologies are integrated, wearable electronic devices linked to existing electronic devices and providing new types of functions and/or services, so-called wearable devices, have been proposed. The wearable device can provide various information related to a user from the viewpoint that the wearable device is worn or attached to the user's body and operated. For example, the wearable device can provide user's biometric information by using a sensor, and provide the biometric information through various interfaces. In addition, the wearable device can share the biometric information with the linked electronic device, thereby supporting the provision of the biometric information through various platforms.

The above information is presented as background information only to assist with an understanding of the disclosure. No determination has been made, and no assertion is made, as to whether any of the above might be applicable as prior art with regard to the disclosure.

### SUMMARY

Aspects of the disclosure are to address at least the above-mentioned problems and/or disadvantages and to provide at least the advantages described below. Accordingly, an aspect of the disclosure is to provide a method for providing biometric information and an electronic device supporting the same.

Additional aspects will be set forth in part in the description which follows and, in part, will be apparent from the description, or may be learned by practice of the presented embodiments.

In accordance with an aspect of the disclosure, a wearable device is provided. The wearable device includes at least one sensor, a communication interface, at least one processor, and memory, comprising one or more storage media, storing instructions, herein the instructions, when executed by the at least one processor individually or collectively, cause the wearable device to obtain first data used to calculate a user's biometric level by using the at least one sensor, transmit the first data to an external electronic device by using the communication interface, receive guide information related to a change in at least part of the biometric level from the external electronic device by using the communication interface, based on the transmission of the first data, obtain second data related to the properties of the guide information by using the at least one sensor, based on the reception of the guide information, and transmit the second data to the external electronic device by using the communication interface.

In accordance with another aspect of the disclosure, an electronic device included in a vehicle is provided. The electronic device included in a vehicle includes a communication interface, a display, an audio interface, at least one processor, and memory, comprising one or more storage media, storing instructions, wherein the instructions, when executed by the at least one processor individually or collectively, cause the electronic device to receive first data from an external electronic device by using the communication interface, calculate a user's biometric level by using the first data, output the calculated user's biometric level by using at least one of the display and the audio interface, output guide information related to a change in at least part of the biometric level by using at least one of the display and the audio interface, based on determining that a specified condition is satisfied, transmit the guide information to the external electronic device by using the communication interface, receive second data from the external electronic device by using the communication interface, based on the transmission of the guide information, generate feedback information about the guide information by using the second data, and output the generated feedback information by using at least one of the display and the audio interface.

In accordance with another aspect of the disclosure, a method for providing biometric information in a wearable device and an electronic device included in a vehicle is provided. The method includes obtaining, by the wearable device, first data used to calculate a user's biometric level by using at least one sensor, and transmitting the first data to the electronic device by using a first communication interface, calculating, by the electronic device, the user's biometric level by using the first data, based on receiving the first data from the wearable device by using a second communication interface, outputting, by the electronic device, the calculated user's biometric level by using at least one of a display and an audio interface, outputting, by the electronic device, guide information related to a change in at least part of the biometric level by using at least one of the display and the audio interface, based on determining that a specified condition is satisfied, and transmitting the guide information to the wearable device by using the second communication interface, obtaining, by the wearable device, second data related to the properties of the guide information by using the at least one sensor, based on receiving the guide information from the electronic device by using the first communication interface, and transmitting the second data to the electronic device by using the first communication interface, and generating, by the electronic device, feedback information about the guide information by using the second data, based on receiving the second data from the wearable device by using the second communication interface, and outputting the feedback information by using at least one of the display and the audio interface.

In accordance with another aspect of the disclosure, one or more non-transitory computer-readable storage media storing one or more computer programs including computer-executable instructions that, when executed by one or more processors of a wearable device individually or collectively, cause the wearable device to perform operations are provided. The operations include obtaining first data used to calculate a user's biometric level by using at least one sensor of the wearable device, transmitting the first data to an external electronic device, receiving guide information related to a change in at least part of the biometric level from the external electronic device, based on the transmission of the first data, obtaining second data related to properties of the guide information by using the at least one sensor, based on the reception of the guide information, and transmitting the second data to the external electronic device.

In accordance with another aspect of the disclosure, one or more non-transitory storage media computer-executable instructions that, when executed by one or more processors of a wearable device individually or collectively, cause the wearable device to perform operations are provided. The operations include obtaining, by the wearable device, first data used to calculate a user's biometric level by using at least one sensor, and transmitting the first data to the electronic device by using a first communication interface, calculating, by the electronic device, the user's biometric level by using the first data, based on receiving the first data from the wearable device by using a second communication interface, outputting, by the electronic device, the calculated user's biometric level information by using at least one of a display and an audio interface, outputting, by the electronic device, guide information related to a change in at least part of the biometric level by using at least one of the display and the audio interface, based on determining that a specified condition is satisfied, and transmitting the guide information to the wearable device by using the second communication interface, obtaining, by the wearable device, second data related to the properties of the guide information by using the at least one sensor, based on receiving the guide information from the electronic device by using the first communication interface, and transmitting the second data to the electronic device by using the first communication interface, and generating, by the electronic device, feedback information about the guide information by using the second data, based on receiving the second data from the wearable device by using the second communication interface, and outputting the feedback information by using at least one of the display and the audio interface.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a diagram illustrating an electronic device in a network environment according to an embodiment of the disclosure;

FIG. 2 is a diagram illustrating components of each of a first electronic device and a second electronic device according to an embodiment of the disclosure;

FIG. 3 is a diagram illustrating an example of an operation flow between a first electronic device and a second electronic device according to an embodiment of the disclosure;

FIG. 4 is a diagram illustrating an example of data processed by a first electronic device according to an embodiment of the disclosure;

FIG. 5 is a diagram illustrating an example of biometric level information output by a second electronic device according to an embodiment of the disclosure;

FIG. 6 is a diagram illustrating an example of guide information output by a second electronic device according to an embodiment of the disclosure;

FIG. 7 is a diagram illustrating an example of an operation flow between a first electronic device and a second electronic device according to an embodiment of the disclosure;

FIG. 8 is a diagram illustrating an example of feedback information output by a second electronic device according to an embodiment of the disclosure; and

FIG. 9 is a diagram illustrating an example of feedback information output by a second electronic device according to an embodiment of the disclosure.

Throughout the drawings, like reference numerals will be understood to refer to like parts, components, and structures.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

When a wearable device shares user's biometric information with an electronic device linked to the wearable device, the wearable device may process raw data obtained through a sensor, generate meaningful biometric information, and transmit the biometric information to the electronic device through a communication interface. However, in such cases, at least part of the biometric information may be lost and/or damaged during a communication process, or may be exposed to hacking issues, so user's personal information included in the biometric information may be vulnerable to security.

In addition, the electronic device may provide biometric information received from the wearable device, through various interfaces, and this may be merely an operation of simply mirroring the biometric information generated by the wearable device. Accordingly, technological advancement capable of improving a user experience for a health service being based on the biometric information through the wearable device and the electronic device is required.

Embodiments of the disclosure may provide a method for providing biometric information and a wearable device and an electronic device supporting the same, capable of preventing security accidents regarding user's personal information included in the biometric information, by excluding direct transmission of the biometric information from the wearable device to the electronic device, when the biometric information capable of being generated based on data obtained by the wearable device is provided through the electronic device linked to the wearable device.

In addition, embodiments of the disclosure may provide a method for providing biometric information and a wearable device and an electronic device supporting the same, capable of improving a user experience for a health service being based on biometric information, by allowing the wearable device and the electronic device to interact with each other in relation to improving the biometric information.

Hereinafter, embodiments of the disclosure are described with reference to the accompanying drawings. However, it is to be understood that this is not intended to limit the disclosure to a specific embodiment form, and includes various modifications, equivalents, and/or alternatives of the embodiments of the disclosure.

Hereinafter, embodiments of the disclosure are described in detail with reference to the drawings so that those skilled in the art to which the disclosure pertains may easily embody the disclosure. However, the disclosure may be implemented in various different forms and is not limited to the embodiments described herein. In relation to the description of the drawings, the same or similar reference numerals may be used for the same or similar components. In addition, in the drawings and related descriptions, descriptions of well-known functions and constructions may be omitted for clarity and conciseness.

It should be appreciated that the blocks in each flowchart and combinations of the flowcharts may be performed by one or more computer programs which include instructions. The entirety of the one or more computer programs may be stored in a single memory device or the one or more computer programs may be divided with different portions stored in different multiple memory devices.

Any of the functions or operations described herein can be processed by one processor or a combination of processors. The one processor or the combination of processors is circuitry performing processing and includes circuitry like an application processor (AP, e.g. a central processing unit (CPU)), a communication processor (CP, e.g., a modem), a graphics processing unit (GPU), a neural processing unit (NPU) (e.g., an artificial intelligence (AI) chip), a wireless fidelity (Wi-Fi) chip, a Bluetooth® chip, a global positioning system (GPS) chip, a near field communication (NFC) chip, connectivity chips, a sensor controller, a touch controller, a finger-print sensor controller, a display driver integrated circuit (IC), an audio CODEC chip, a universal serial bus (USB) controller, a camera controller, an image processing IC, a microprocessor unit (MPU), a system on chip (SoC), an IC, or the like.

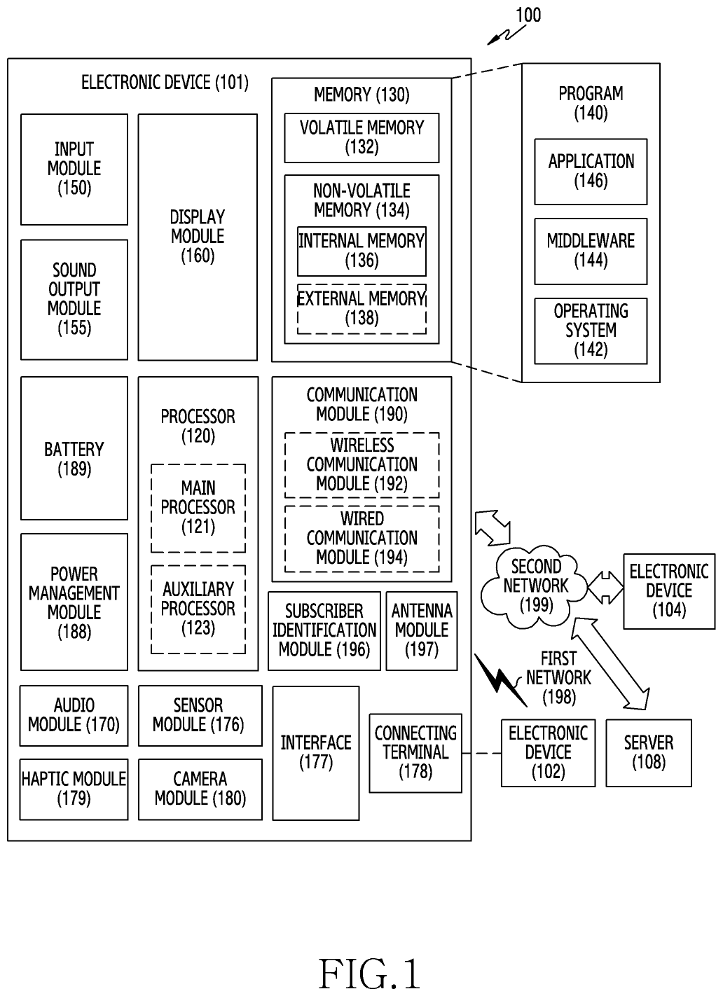

FIG. 1 is a block diagram illustrating an electronic device 101 in a network environment 100 according to an embodiment of the disclosure.

Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In some embodiments, at least one of the components (e.g., the connecting terminal 178) may be omitted from the electronic device 101, or one or more other components may be added in the electronic device 101. In some embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to one embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thererto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to one embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a legacy cellular network, a fifth generation (5G) network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a fourth generation (4G) network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the mmWave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element composed of a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to various embodiments, the antenna module 197 may form a mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

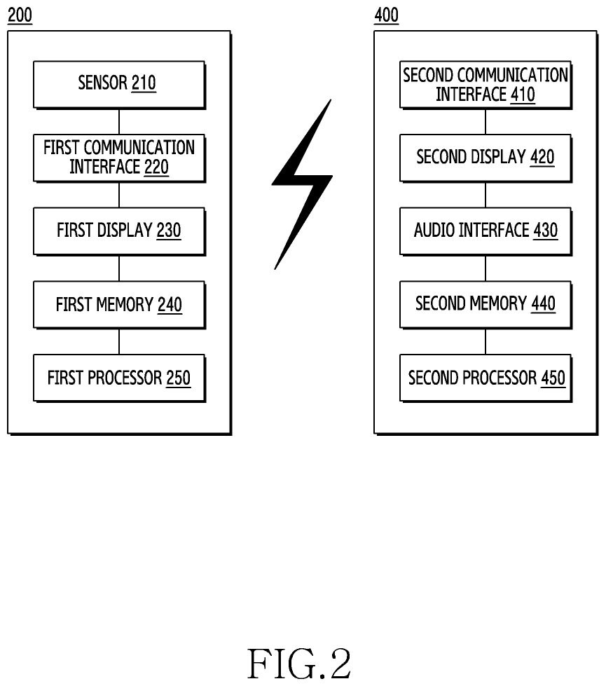

FIG. 2 is a diagram illustrating components of each of a first electronic device and a second electronic device according to an embodiment of the disclosure.

Referring to FIG. 2, a first electronic device 200 and a second electronic device 400 of an embodiment may interact with each other (e.g., establish communication, transmit and receive data, and/or transmit and receive information) and provide user's biometric level information. For example, the first electronic device 200 may obtain first data used to generate the biometric level information and transmit the first data to the second electronic device 400, and the second electronic device 400 may generate and output the biometric level information by using the first data received from the first electronic device 200. In addition, the first electronic device 200 and the second electronic device 400 may interact with each other and change at least part of the biometric level information (or improve a user's biometric level indicated by the biometric level information). For example, the first electronic device 200 may obtain second data, based on guide information related to a change in at least part of the biometric level information, received from the second electronic device 400, and transmit the second data to the second electronic device 400, and the second electronic device 400 may generate and output feedback information about the guide information by using the second data received from the first electronic device 200.

In an embodiment, the first electronic device 200 may include a wearable device (e.g., smart watch, smart band, and/or smart glasses) that may be worn or attached to the user's body and operated. In various embodiments, the first electronic device 200 may also include a device having a different aspect from the wearable device as long as the first electronic device 200 includes the functions, components, or mechanisms described below.

According to an embodiment, the first electronic device 200 may include at least one sensor 210, a first communication interface 220, a first display 230, a first memory 240, and a first processor 250. In various embodiments, the first electronic device 200 may omit at least some of the components described above, or may further include other additional components. For example, the first electronic device 200 may further include at least some of the components of the electronic device (e.g., electronic device 101 of FIG. 1) mentioned through FIG. 1.

In an embodiment, the at least one sensor 210 may output an electrical signal or data corresponding to at least one of a state of the first electronic device 200 with respect to an external environment and a physical state of a user. For example, the at least one sensor 210 may include at least one of an acceleration sensor, a gyro sensor, and a global positioning system (GPS) sensor, and may output an electrical signal or data for at least one of movement, position, direction, and acceleration of the first electronic device 200 according to a specified sampling rate. In addition, for example, the at least one sensor 210 may include a photoplethysmography (PPG) sensor, and may output an electrical signal or data for at least one of a heart rate (HR) and an inter-beat interval (IBI) of the user's body.

In an embodiment, the first communication interface 220 may support communication between the first electronic device 200 and at least one external device (e.g., second electronic device 400). For example, the first communication interface 220 may establish a communication channel with at least one external device according to a prescribed protocol, and may transmit and receive at least one of a signal, data, and information through the communication channel. In various embodiments, the first communication interface 220 may include at least one of an interface (e.g., wireless communication module 192 of FIG. 1) supporting wireless communication (e.g., Wi-Fi, Wi-Fi direct, and/or Bluetooth) and an interface (e.g., wired communication module 194 of FIG. 1 and/or USB interface) supporting wired communication.

In an embodiment, the first display 230 may be at least partially exposed to the outside of the first electronic device 200 and visually provide various contents. For example, the first display 230 may display a user interface including various information (e.g., time information, date information, weather information, and/or battery information) in a sleep state (or low power state) of the first electronic device 200. In addition, for example, the first display 230 may display a user interface including an execution screen of an application program in an active state of the first electronic device 200.

In an embodiment, the first memory 240 may store at least one data related to the operation of the first electronic device 200, or may store instructions related to the functional operation of components of the first electronic device 200. For example, the first memory 240 may at least temporarily store data output from the at least one sensor 210. In addition, for example, the first memory 240 may at least temporarily store data or information received from at least one external device via the first communication interface 220. In addition, for example, the first memory 240 may store at least one application program supporting the provision of a health service.

In an embodiment, the first processor 250 (e.g., processor including a processing circuit) may execute codes (e.g., instructions) of a programming language stored in the first memory 240 and control various functions or operations of the first electronic device 200. For example, the first processor 250 may be electrically connected to components of the first electronic device 200 and may transfer (or transmit) at least one command, signal, or data involved in performing a function or operation of the first electronic device 200 to the relevant components. In various embodiments, the first processor 250 may include at least one of a central processing unit, an application processor, and a communication processor.

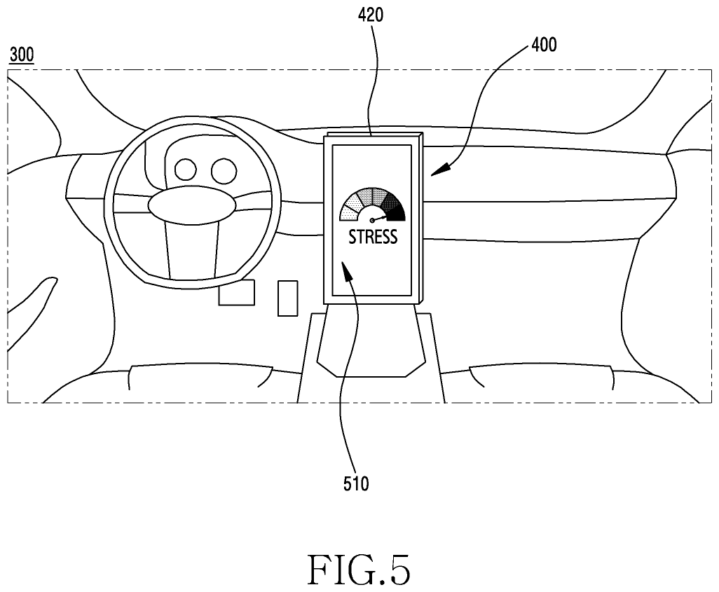

In an embodiment, the second electronic device 400 may include an electronic device arranged in at least one area inside a vehicle (e.g., vehicle 300 of FIGS. 5, 6, 8, and/or 9). For example, the second electronic device 400 may be arranged in a center fascia area or dashboard area of the vehicle 300. In addition, for example, the second electronic device 400 may be implemented as a vehicle system (e.g., audio video navigation (AVN) system and/or in vehicle infotainment (IVI) system) mounted on the vehicle 300 itself.

In an embodiment, the second electronic device 400 may include a second communication interface 410, a second display 420, an audio interface 430, a second memory 440, and a second processor 450. In various embodiments, the second electronic device 400 may omit at least some of the components described above, or may further include other additional components. For example, the second electronic device 400 may further include at least some of the components of the electronic device 101 mentioned through FIG. 1.

In an embodiment, the second communication interface 410 may support communication between the second electronic device 400 and at least one external device (e.g., first electronic device 200). For example, the second communication interface 410 may establish a communication channel with at least one external device according to a prescribed protocol, and may transmit and receive at least one of a signal, data, and information through the communication channel. In various embodiments, the second communication interface 410 may include at least one of an interface (e.g., wireless communication module 192 of FIG. 1) supporting wireless communication (e.g., Wi-Fi, Wi-Fi direct, and/or Bluetooth) and an interface (e.g., wired communication module 194 of FIG. 1 and/or USB interface) supporting wired communication.

In an embodiment, the second display 420 may be at least partially exposed to the outside of the second electronic device 400 and visually provide various contents. For example, the second display 420 may display a user interface including various information (e.g., speed information and/or driving route information) related to driving of the vehicle 300. In addition, for example, the second display 420 may display a user interface including various information (e.g., user's biometric level information, guide information related to a change in at least part of the biometric level, and/or feedback information about the guide information) that are generated by the second electronic device 400, based on data received from at least one external device.

In an embodiment, the audio interface 430 may be at least partially exposed to the outside of the second electronic device 400 and audibly provide various audio signals. For example, the audio interface 430 may convert various information (e.g., user's biometric level information, guide information related to a change in at least part of the biometric level, and/or feedback information about the guide information) generated by the second electronic device 400 into a voice signal of an audible frequency band, and output the voice signal. In addition, for example, the audio interface 430 may receive a voice signal of a user speech responding to the output voice signal. According to various embodiments, the audio interface 430 may include at least one of a speaker and a receiver.

In an embodiment, the second memory 440 may store at least one data related to the operation of the second electronic device 400, or store instructions related to the functional operation of components of the second electronic device 200. For example, the second memory 440 may at least temporarily store data related to various information (e.g., user's biometric level information, guide information related to a change in at least part of the biometric level, and/or feedback information about the guide information) generated by the second electronic device 400. In addition, for example, the second memory 440 may store at least one of an algorithm, a mathematical formula, and an application program that may be used to generate the user's biometric level information, based on data received from at least one external device.

In an embodiment, the second processor 450 (e.g., processor including a processing circuit) may execute codes (e.g., instructions) of a programming language stored in the second memory 440 and control various functions or operations of the second electronic device 400. For example, the second processor 450 may be electrically connected to components of the second electronic device 400, and may transfer (or transmit) at least one command, signal, or data involved in performing a function or operation of the second electronic device 400 to the relevant components. In various embodiments, the second processor 450 may include at least one of a central processing unit, an application processor, and a communication processor.

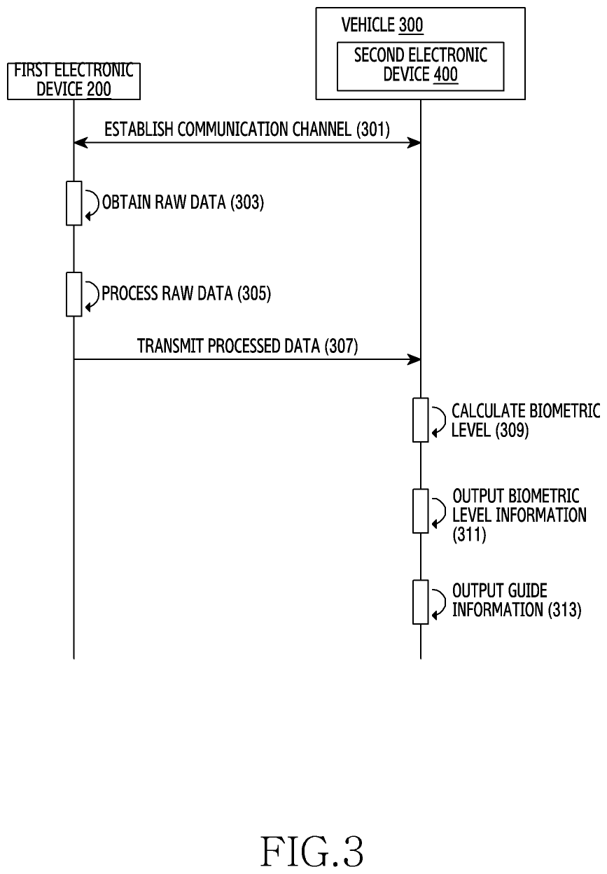

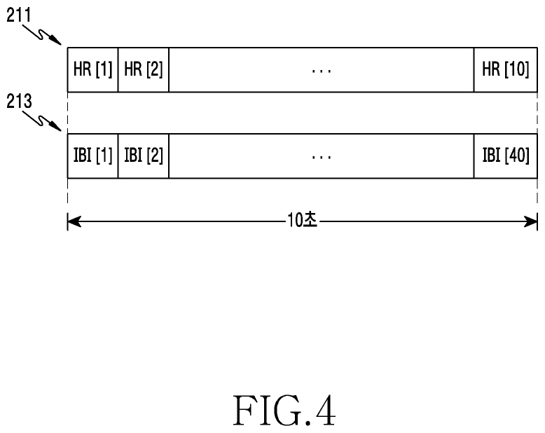

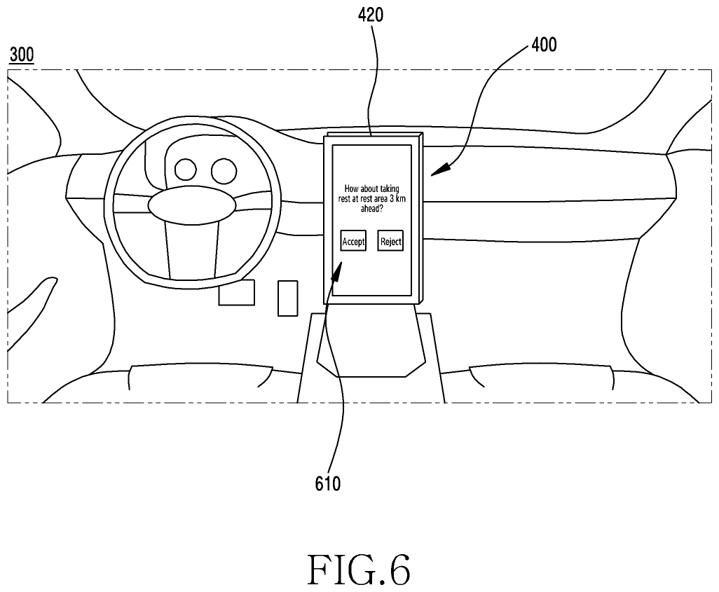

FIG. 3 is a diagram illustrating an example of an operation flow between a first electronic device and a second electronic device according to an embodiment of the disclosure. FIG. 4 is a diagram illustrating an example of data processed by the first electronic device according to an embodiment of the disclosure. FIG. 5 is a diagram illustrating an example of biometric level information output by the second electronic device according to an embodiment of the disclosure. FIG. 6 is a diagram illustrating an example of guide information output by the second electronic device according to an embodiment of the disclosure. Hereinafter, when the embodiment of FIG. 3 is explained, the embodiments of FIGS. 4, 5, and 6 may be referred to together.

Referring to FIG. 3, in the embodiment below, each operation may be performed sequentially, but is not necessarily performed sequentially. For example, the order of each operation may be changed as well, and at least two operations may be performed in parallel as well.

According to an embodiment, it may be understood that operations 301 to 313 are performed by a first electronic device 200 when instructions stored in a first memory (e.g., first memory 240 of FIG. 2) of the first electronic device (e.g., first electronic device 200 of FIG. 2) are executed by a first processor (e.g., first processor 250 of FIG. 2), or are performed by a second electronic device 400 when instructions stored in a second memory (e.g., second memory 440 of FIG. 2) of the second electronic device (e.g., second electronic device 400 of FIG. 2) are executed by a second processor (e.g., second processor 450 of FIG. 2).

Referring to FIG. 3, in operation 301, a first electronic device 200 (e.g., wearable device), and a second electronic device 400 included in a vehicle 300, may establish a communication channel. For example, each of the first electronic device 200 and the second electronic device 400 may be wiredly connected to each other based on an interface (e.g., wired communication module 194 of FIG. 1 and/or USB interface) supporting wired communication. In addition, for example, each of the first electronic device 200 and the second electronic device 400 may be wirelessly connected to each other based on an interface (e.g., wireless communication module 192 of FIG. 1) supporting wireless communication. According to various embodiments related to the wireless connection, the first electronic device 200 may search for at least one external device capable of wireless connection with the first electronic device 200, and display a list including identification information of the searched at least one external device by using a first display (e.g., first display 230 of FIG. 2). The first electronic device 200 may request a wireless communication connection to the second electronic device 400, based on a user input of selecting identification information of the second electronic device 400 (or vehicle 300) from the list. In various embodiments, in response to the establishment of the communication channel, the first electronic device 200 (or first processor 250) and the second electronic device 400 (or second processor 450) may establish various rules (or policies) related to the provision of user's biometric level information. For example, the first electronic device 200 may transmit, to the second electronic device 400, a signal and/or data indicating at least one of the type (e.g., HR data and/or IBI data) of data to be provided, the format (e.g., data of a structure processed from raw data) of the data, and a transmission period of the data, together with the identification information of the first electronic device 200. For another example, the first electronic device 200 may receive a signal and/or data requesting information about at least one of the type of the data, the format of the data, and the transmission period of the data, from the second electronic device 400. In various embodiments, the transmission/reception period of the data may be adaptively determined based on at least one of a power state of the first electronic device 200 and/or the second electronic device 400, the performance thereof, and the version of an application program supporting the provision of a health service.

In operation 303, the first electronic device 200 may obtain at least one raw data. For example, the first electronic device 200 may obtain raw data that is output according to a specified sampling rate from a PPG sensor included in at least one sensor (e.g., sensor 210 of FIG. 2).

In operations 305 and 307, the first electronic device 200 may process the obtained raw data, and transmit the processed data to the second electronic device 400. For example, referring to FIG. 4, the first electronic device 200 may process the raw data and generate first data (e.g., HR data 211 and/or IBI data 213) that may be used to calculate a user's biometric level (or generate biometric level information). The first electronic device 200 may transmit a packet including a specified amount (e.g., ten HR data 211 and/or forty IBI data 213) of the first data to the second electronic device 400 according to a specified period (e.g., 10-second period) by using a first communication interface (e.g., first communication interface 220 of FIG. 2). According to various embodiments, the amount of the first data included in the packet may be adaptively determined based on at least one of a power state of the first electronic device 200 and the size of the packet, or may be determined according to a default value that is set to the first electronic device 200.

In an embodiment, the above-described operations 303, 305, and 307 may be repeatedly performed according to the specified period (e.g., 10-second period) for transmitting the packet including the first data to the second electronic device 400.

According to an embodiment, the first electronic device 200 may, at an operation (or time) of initially transmitting the packet, obtain third data (e.g., resting HR data generated based on raw data output from a PPG sensor when a user is in a stable state) recorded in an application program (e.g., application program supporting the provision of a health service) stored in a first memory (e.g., first memory 240 of FIG. 2), and transmit the third data together with the packet (or included in the packet) to the second electronic device 400. Alternatively, the first electronic device 200 may, at an operation (or time) of establishing a communication channel between the first electronic device 200 and the second electronic device 400, obtain third data from the application program, and transmit the third data to the second electronic device 400.

In operation 309, the second electronic device 400 may receive the first data (or packet including the first data) transmitted from the first electronic device 200 by using a second communication interface (e.g., second communication interface 410 of FIG. 2), and calculate a user's biometric level by using the first data. For example, the second electronic device 200 may apply the first data to at least one of an algorithm, a mathematical formula, and an application program stored in a second memory (e.g., second memory 440 of FIG. 2), and calculate a biometric level including at least one of a user's stress level and sleepiness level.

In operation 311, the second electronic device 400 may generate user's biometric level information, based on the generated biometric level, and output the generated biometric level information. For example, the second electronic device 400 may calculate a reference biometric level, based on the third data (e.g., resting HR data) received from the first electronic device 200 by using the second communication interface 410, and generate biometric level information indicating the size (or numerical value) of at least one of a user's stress level and sleepiness level, based on a comparison between the biometric level calculated according to the execution of operation 309 and the reference biometric level. In addition, for example, referring to FIG. 5, the second electronic device 400 may display a user interface 510 including at least one of an image (e.g., icon, symbol, and/or graph) and text visually representing the generated biometric level information, by using the second display 420. In addition, for example, the second electronic device 400 may convert the generated biometric level information into a voice signal of an audible frequency band, and output the voice signal through an audio interface (e.g., audio interface 430 of FIG. 2). In various embodiments, the biometric level information may be output visually and audibly through a combination of the second display 420 and the audio interface 430, or may be output visually or audibly through either the second display 420 or the audio interface 430, based on a user setting for the second electronic device 400.

In operation 313, after outputting the biometric level information, the second electronic device 400 may determine whether a specified condition is satisfied with the biometric level information, and output guide information, based on the determination that the specified condition is satisfied. For example, the second electronic device 400 may determine that the specified condition is satisfied when the size (or numerical value) of at least one of a user's stress level and sleepiness level indicated by the biometric level information is maintained above a specified threshold value for a specified time (e.g., one minute). In addition, for example, referring to FIG. 6, the second electronic device 400 may display a user interface 610 including an image and/or text visually representing guide information for changing at least part of the user's biometric level, by using the second display 420, based on the determination that the specified condition is satisfied. According to an embodiment, the guide information may include a request (e.g., request for a user to rest (visit a rest area and/or a rest stop)) of inducing a change (e.g., decrease) in at least one of the user's stress level and sleepiness level indicated by the biometric level information. In addition, for example, the second electronic device 400 may convert the guide information into a voice signal of an audible frequency band, and output the voice signal through the audio interface 430.

In various embodiments, the output of the guide information by the second electronic device 400 may be omitted. For example, the second electronic device 400 may not output the guide information when it is determined that the specified condition is not satisfied with the biometric level information.

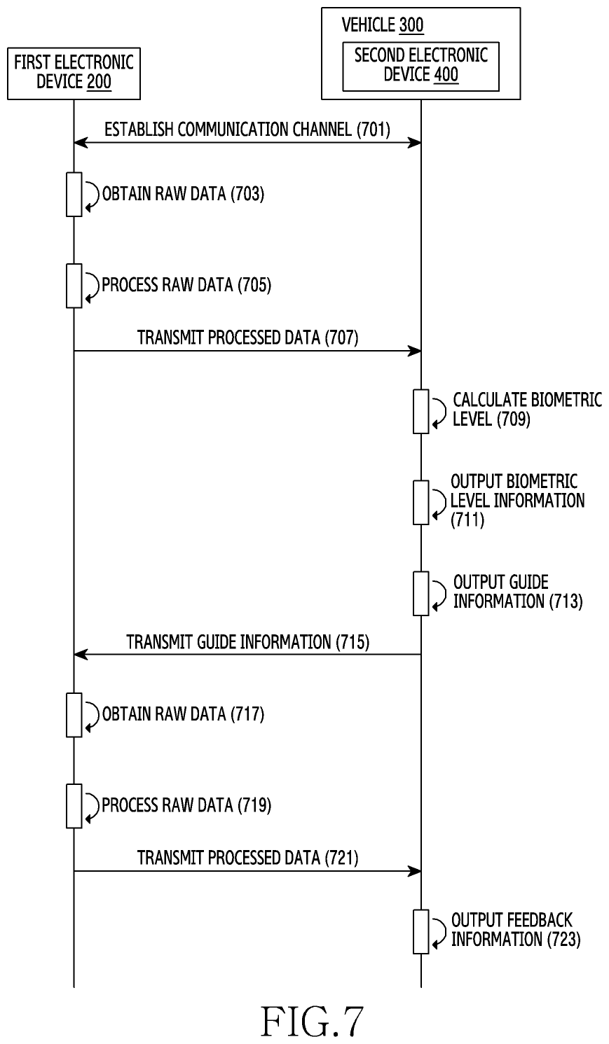

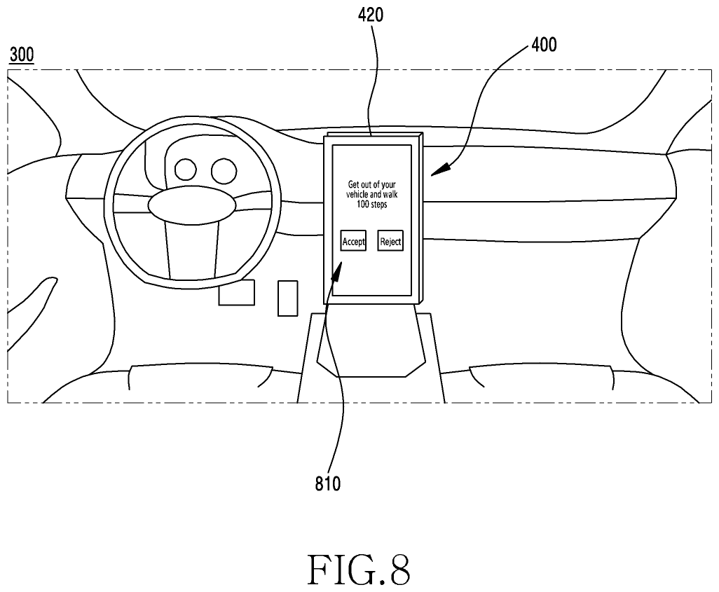

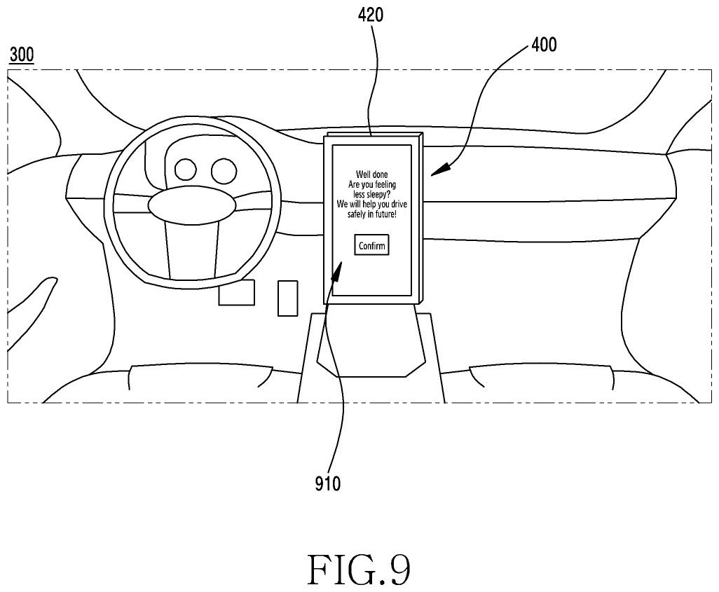

FIG. 7 is a diagram illustrating an example of an operation flow between a first electronic device and a second electronic device according to an embodiment of the disclosure. FIG. 8 is a diagram illustrating an example of feedback information output by the second electronic device according to an embodiment of the disclosure. FIG. 9 is a diagram illustrating an example of feedback information output by the second electronic device according to an embodiment of the disclosure. Hereinafter, when the embodiment of FIG. 7 is explained, the embodiments of FIGS. 8 and 9 may be referred to together.

Referring to FIG. 7, in the embodiments below, each operation may be performed sequentially, but is not necessarily performed sequentially. For example, the order of each operation may be changed as well, and at least two operations may be performed in parallel as well.

According to an embodiment, it may be understood that operations 701 to 723 are performed by a first electronic device 200 when instructions stored in a first memory (e.g., first memory 240 of FIG. 2) of the first electronic device (e.g., first electronic device 200 of FIG. 2) are executed by a first processor (e.g., first processor 250 of FIG. 2), or are performed by a second electronic device 400 when instructions stored in a second memory (e.g., second memory 440 of FIG. 2) of the second electronic device (e.g., electronic device 400 of FIG. 2) are executed by a second processor (e.g., second processor 450 of FIG. 2).

In the embodiment of FIG. 7, each of operations 701, 703, 705, 707, 709, 711, and 713 may correspond to each of operations 301, 303, 305, 307, 309, 311, and 313 described above through the embodiment of FIG. 3. For example, in operation 701, the first electronic device 200 and the second electronic device 400 may establish a communication channel, and may establish various rules (or policies) (e.g., type of data to be transmitted and received, format of the data, and/or transmission and reception period of the data) related to the provision of user's biometric level information by using the communication channel. In addition, for example, in operations 703, 705, and 707, the first electronic device 200 may process raw data obtained from a PPG sensor included in at least one sensor (e.g., sensor 210 of FIG. 2) and generate first data (e.g., HR data 211 and/or IBI data 213 of FIG. 4) that may be used to calculate a user's biometric level (or generate biometric level information), and may transmit the first data (or packet including the first data) to the second electronic device 400 at a specified period (e.g., 10-second period) by using a first communication interface (e.g., first communication interface 220 of FIG. 2). In addition, for example, in operations 709 and 711, the second electronic device 400 may apply the first data received from the first electronic device 200 by using a second communication interface (e.g., second communication interface 410 of FIG. 2), to at least one of an algorithm, a mathematical formula, and an application program stored in a second memory (e.g., second memory 440 of FIG. 2), and calculate a biometric level including at least one of a user's stress level and a user's sleepiness level, and may generate biometric level information indicating the size (or numerical value) of the calculated biometric level and output the biometric level information through at least one of a second display (e.g., second display 420 of FIG. 2) and an audio interface (e.g., audio interface 430 of FIG. 2). In addition, for example, referring to FIG. 8, in operation 713, the second electronic device 400 may display a user interface 810 including guide information related to a request (e.g., request for a user's activity (walking more than a specified number of steps)) of inducing a change (e.g., decrease) in the size of the user's biometric level (e.g., stress level and/or sleepiness level) by using the second display 420, based on the determination that the biometric level information satisfies a specified condition.

Although not shown, according to various embodiments, the second electronic device 400 may control at least some functions of a vehicle 300 in which a user is riding, based on the determination that the biometric level information satisfies a specified condition. For example, the second electronic device 400 may change a driving mode of the vehicle 300 to a low-speed driving mode, or change a driving route of the vehicle 300 displayed through the second display 420 to a driving route with a smooth traffic flow (e.g., driving route with less vehicles, driving route with construction sites excluded, and/or driving route with curve courses minimized).

Referring to FIG. 7, in operation 715, the second electronic device 400 may transmit the guide information to the first electronic device 200 by using the second communication interface 410, based on the outputting of the guide information.

In operation 717, the first electronic device 200 may receive the guide information by using the first communication interface 220, and obtain raw data related to the properties of the guide information. For example, the first electronic device 200 may analyze a keyword of the request included in the guide information, determine the properties of the request, and obtain (or receive) raw data corresponding to the properties of the request from at least one sensor (e.g., sensor 210 of FIG. 1). For example, when the properties of the request included in the guide information indicate a user's visit to a specific location, the first electronic device 200 may obtain (or receive) raw data that is output according to a specified sampling rate from a GPS sensor included in the at least one sensor 210. For another example, when the properties of the request included in the guide information indicate user's walking, the first electronic device 200 may obtain (or receive) raw data that is output according to a specified sampling rate from an acceleration sensor included in the at least one sensor 210.

In operations 719 and 721, the first electronic device 200 may process the obtained raw data, and transmit the processed data to the second electronic device 400. For example, the first electronic device 200 may process the raw data corresponding to the properties of the request included in the guide information, generate second data indicating a location of the user (or first electronic device 200) or the number of steps of the user, and transmit the second data to the second electronic device 400 by using the first communication interface 220.

In operation 723, the second electronic device 400 may generate feedback information by using the second data received based on the second communication interface 410, and output the generated feedback information. For example, referring to FIG. 9, the second electronic device 400 may determine whether the user has performed the request included in the guide information, based on location information of the user (or first electronic device 200) or step count information of the user indicated by the second data, and may display a user interface 910 including an image and/or text for expressing feedback information indicating a response corresponding to the determination on whether the user has performed, by using the second display 420. In addition, for example, the second electronic device 400 may convert the generated feedback information into a voice signal of an audible frequency band, and output the voice signal through an audio interface (e.g., audio interface 430 of FIG. 2).

A wearable device of an embodiment of the disclosure may include at least one sensor, a communication interface, at least one processor, and memory storing instructions.

According to an embodiment, the instructions may, when executed by the at least one processor, cause the wearable device to obtain first data used to calculate a user's biometric level by using the at least one sensor, transmit the first data to an external electronic device by using the communication interface, receive guide information related to a change in at least part of the biometric level from the external electronic device by using the communication interface, based on the transmission of the first data, obtain second data related to the properties of the guide information by using the at least one sensor, based on the reception of the guide information, and transmit the second data to the external electronic device by using the communication interface.

According to an embodiment, the external electronic device may include an electronic device arranged in at least one area inside a vehicle.

According to an embodiment, the at least one sensor may include a photoplethysmography (PPG) sensor, and the instructions may, when executed by the at least one processor, cause the wearable device to process raw data output from the PPG sensor and obtain the first data including at least one of heart rate (HR) data and inter-beat interval (IBI) data.

According to an embodiment, the instructions may, when executed by the at least one processor, cause the wearable device to transmit the first data to the external electronic device, based on a specified period.

According to an embodiment, the instructions may, when executed by the at least one processor, cause the wearable device to transmit third data including resting HR data recorded in an application program stored in the memory to the external electronic device by using the communication interface, at a time of establishing communication with the external electronic device by using the communication interface or at a time of initially transmitting the first data to the external electronic device based on the specified period.

According to an embodiment, the instructions may, when executed by the at least one processor, cause the wearable device to receive the guide information including at least one request related to a decrease in the size of the biometric level from the external electronic device by using the communication interface, and analyze a keyword corresponding to the at least one request and determine the properties of the guide information.

According to an embodiment, the at least one sensor may include at least one of an acceleration sensor and a GPS sensor, and the instructions may, when executed by the at least one processor, cause the wearable device to process raw data output from at least one of the acceleration sensor and the GPS sensor and obtain the second data including at least one of location data of the wearable device and step count data of a user corresponding to the properties of the guide information.

An electronic device included in a vehicle of an embodiment of the disclosure may include a communication interface, a display, an audio interface, at least one processor, and memory storing instructions.

According to an embodiment, the instructions may, when executed by the at least one processor, cause the electronic device to receive first data from an external electronic device by using the communication interface, calculate a user's biometric level by using the first data, output the calculated user's biometric level information by using at least one of the display and the audio interface, output guide information related to a change in at least part of the biometric level by using at least one of the display and the audio interface, based on determining that a specified condition is satisfied, transmit the guide information to the external electronic device by using the communication interface, receive second data from the external electronic device by using the communication interface, based on the transmission of the guide information, generate feedback information about the guide information by using the second data, and output the generated feedback information by using at least one of the display and the audio interface.

According to an embodiment, the instructions may, when executed by the at least one processor, cause the electronic device to apply the first data to at least one of an algorithm, a mathematical formula, and an application program stored in the memory and calculate the user's biometric level including at least one of a stress level and a sleepiness level.

According to an embodiment, the instructions may, when executed by the at least one processor, cause the electronic device to determine that the specified condition is satisfied, based on the size of the user's biometric level being maintained above a specified threshold value for a specified time.

According to an embodiment, the instructions may, when executed by the at least one processor, cause the electronic device to output the guide information including at least one request related to a decrease in the size of the user's biometric level.

According to an embodiment, the instructions may, when executed by the at least one processor, cause the electronic device to determine whether a user has performed the at least one request, based on the second data, and generate the feedback information including a response corresponding to whether the user has performed.

According to an embodiment, the instructions may, when executed by the at least one processor, cause the electronic device to receive the second data including at least one of location data of the external electronic device and step count data of a user from the external electronic device by using the communication interface.

According to an embodiment, the instructions may, when executed by the at least one processor, cause the electronic device to control a change in at least one of a driving mode and driving route of the vehicle, based on determining that the specified condition is satisfied.

A method for providing biometric information in a wearable device and an electronic device included in a vehicle of an embodiment of the disclosure may include obtaining, by the wearable device, first data used to calculate a user's biometric level by using at least one sensor, and transmitting the first data to the electronic device by using a first communication interface, calculating, by the electronic device, the user's biometric level by using the first data, based on receiving the first data from the wearable device by using a second communication interface, outputting, by the electronic device, the calculated user's biometric level information by using at least one of a display and an audio interface, outputting, by the electronic device, guide information related to a change in at least part of the biometric level by using at least one of the display and the audio interface, based on determining that a specified condition is satisfied, and transmitting the guide information to the wearable device by using the second communication interface, obtaining, by the wearable device, second data related to the properties of the guide information by using the at least one sensor, based on receiving the guide information from the electronic device by using the first communication interface, and transmitting the second data to the electronic device by using the first communication interface, and generating, by the electronic device, feedback information about the guide information by using the second data, based on receiving the second data from the wearable device by using the second communication interface, and outputting the feedback information by using at least one of the display and the audio interface.

According to an embodiment, obtaining the first data may include processing, by the wearable device, raw data output from a photoplethysmography (PPG) sensor included in the at least one sensor and obtaining the first data including at least one of heart rate (HR) data and inter-beat interval (IBI) data.

According to an embodiment, obtaining the second data may include processing, by the wearable device, raw data output from at least one of an acceleration sensor and a GPS sensor included in the at least one sensor and obtain the second data including at least one of location data of the wearable device and step count data of a user corresponding to the properties of the guide information.

According to an embodiment, transmitting the guide information to the wearable device may include determining, by the electronic device, that the specified condition is satisfied, based on the size of the user's biometric level being maintained above a specified threshold value for a specified time.

According to an embodiment, outputting the guide information may include outputting, by the electronic device, the guide information including at least one request related to a decrease in the size of the user's biometric level.

According to an embodiment, generating the feedback information may include determining, by the electronic device, whether a user has performed the at least one request, based on the second data, and generating the feedback information including a response corresponding to whether the user has performed.

A non-transitory storage medium readable by a computer recording a computer program for executing a method for providing biometric information of an embodiment of the disclosure may be provided, and the method may include obtaining, by the wearable device, first data used to calculate a user's biometric level by using at least one sensor, and transmitting the first data to the electronic device by using a first communication interface, calculating, by the electronic device, the user's biometric level by using the first data, based on receiving the first data from the wearable device by using a second communication interface, outputting, by the electronic device, the calculated user's biometric level information by using at least one of a display and an audio interface, outputting, by the electronic device, guide information related to a change in at least part of the biometric level by using at least one of the display and the audio interface, based on determining that a specified condition is satisfied, and transmitting the guide information to the wearable device by using the second communication interface, obtaining, by the wearable device, second data related to the properties of the guide information by using the at least one sensor, based on receiving the guide information from the electronic device by using the first communication interface, and transmitting the second data to the electronic device by using the first communication interface, and generating, by the electronic device, feedback information about the guide information by using the second data, based on receiving the second data from the wearable device by using the second communication interface, and outputting the feedback information by using at least one of the display and the audio interface.

According to embodiments of the disclosure, a mechanism capable of providing user's biometric information through interaction between a wearable device and an electronic device may be provided.

In addition, according to embodiments of the disclosure, when biometric information capable of being generated based on data obtained by a wearable device is provided through an electronic device linked to the wearable device, a security accident regarding user personal information included in the biometric information may be prevented by excluding the direct transmission of the biometric information from the wearable device to the electronic device.

In addition, according to embodiments of the disclosure, a wearable device and an electronic device may interact with each other in relation to improvement of biometric information, thereby improving a user experience for a health service being based on the biometric information.

The effects obtainable from the disclosure are not limited to the effects mentioned above, and other effects not mentioned will be clearly understood by those skilled in the art from various embodiments of the disclosure.

The electronic device according to various embodiments may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic devices are not limited to those described above.

It should be appreciated that various embodiments of the disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wiredly), wirelessly, or via a third element.

As used in connection with various embodiments of the disclosure, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

It will be appreciated that various embodiments of the disclosure according to the claims and description in the specification can be realized in the form of hardware, software or a combination of hardware and software.

Any such software may be stored in non-transitory computer readable storage media. The non-transitory computer readable storage media store one or more computer programs (software modules), the one or more computer programs include computer-executable instructions that, when executed by one or more processors of an electronic device individually or collectively, cause the electronic device to perform a method of the disclosure.

Any such software may be stored in the form of volatile or non-volatile storage such as, for example, a storage device like read only memory (ROM), whether erasable or rewritable or not, or in the form of memory such as, for example, random access memory (RAM), memory chips, device or integrated circuits or on an optically or magnetically readable medium such as, for example, a compact disk (CD), digital versatile disc (DVD), magnetic disk or magnetic tape or the like. It will be appreciated that the storage devices and storage media are various embodiments of non-transitory machine-readable storage that are suitable for storing a computer program or computer programs comprising instructions that, when executed, implement various embodiments of the disclosure. Accordingly, various embodiments provide a program comprising code for implementing apparatus or a method as claimed in any one of the claims of this specification and a non-transitory machine-readable storage storing such a program.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. A wearable device comprising:
at least one sensor;
a communication interface;
at least one processor; and
memory, comprising one or more storage media, storing instructions,
wherein the instructions, when executed by the at least one processor individually or collectively, cause the wearable device to:
obtain first data used to calculate a user's biometric level by using the at least one sensor,
transmit the first data to an external electronic device by using the communication interface,
receive guide information related to a change in at least part of the biometric level from the external electronic device by using the communication interface, based on the transmission of the first data,
obtain second data related to properties of the guide information by using the at least one sensor, based on the reception of the guide information, and
transmit the second data to the external electronic device by using the communication interface.

2. The wearable device of claim 1, wherein the external electronic device comprises an electronic device arranged in at least one area inside a vehicle.

3. The wearable device of claim 1,
wherein the at least one sensor comprises a photoplethysmography (PPG) sensor, and
wherein the instructions, when executed by the at least one processor individually or collectively, cause the wearable device to:
process raw data output from the PPG sensor and obtain the first data comprising at least one of heart rate (HR) data and inter-beat interval (IBI) data.

4. The wearable device of claim 1, wherein the instructions, when executed by the at least one processor individually or collectively, cause the wearable device to:
transmit the first data to the external electronic device, based on a specified period.

5. The wearable device of claim 4, wherein the instructions, when executed by the at least one processor individually or collectively, cause the wearable device to:
transmit third data comprising resting HR data recorded in an application program stored in the memory to the external electronic device by using the communication interface, at a time of establishing communication with the external electronic device by using the communication interface or at a time of initially transmitting the first data to the external electronic device based on the specified period.

6. The wearable device of claim 1, wherein the instructions, when executed by the at least one processor individually or collectively, cause the wearable device to:
receive the guide information comprising at least one request related to a decrease in a size of the biometric level from the external electronic device by using the communication interface; and
analyze a keyword corresponding to the at least one request and determine the properties of the guide information.

7. The wearable device of claim 6,
wherein the at least one sensor comprises at least one of an acceleration sensor and a global positioning system (GPS) sensor, and
wherein the instructions, when executed by the at least one processor individually or collectively, cause the wearable device to:
process raw data output from at least one of the acceleration sensor and the GPS sensor and obtain the second data comprising at least one of location data of the wearable device and step count data of a user corresponding to the properties of the guide information.

8. An electronic device comprised in a vehicle, comprising:
a communication interface;
a display;
an audio interface;
at least one processor; and
memory, comprising one or more storage media, storing instructions,
wherein the instructions, when executed by the at least one processor individually or collectively, cause the electronic device to:
receive first data from an external electronic device by using the communication interface,
calculate a user's biometric level by using the first data,
output the calculated user's biometric level by using at least one of the display and the audio interface,
output guide information related to a change in at least part of the biometric level by using at least one of the display and the audio interface, based on determining that a specified condition is satisfied,
transmit the guide information to the external electronic device by using the communication interface,
receive second data from the external electronic device by using the communication interface, based on the transmission of the guide information,
generate feedback information about the guide information by using the second data, and
output the generated feedback information by using at least one of the display and the audio interface.

9. The electronic device of claim 8, wherein the instructions, when executed by the at least one processor individually or collectively, cause the electronic device to:
apply the first data to at least one of an algorithm, a mathematical formula, and an application program stored in the memory and calculate the user's biometric level comprising at least one of a stress level and a sleepiness level.

10. The electronic device of claim 8, wherein the instructions, when executed by the at least one processor individually or collectively, cause the electronic device to:
determine that the specified condition is satisfied, based on a size of the user's biometric level being maintained above a specified threshold value for a specified time.

11. The electronic device of claim 10, wherein the instructions, when executed by the at least one processor individually or collectively, cause the electronic device to:
output the guide information comprising at least one request related to a decrease in the size of the user's biometric level.

12. The electronic device of claim 11, wherein the instructions, when executed by the at least one processor individually or collectively, cause the electronic device to:
determine whether a user has performed the at least one request, based on the second data; and
generate the feedback information comprising a response corresponding to whether the user has performed.

13. The electronic device of claim 8, wherein the instructions, when executed by the at least one processor individually or collectively, cause the electronic device to:
receive the second data comprising at least one of location data of the external electronic device and step count data of a user from the external electronic device by using the communication interface.

14. The electronic device of claim 8, wherein the instructions, when executed by the at least one processor individually or collectively, cause the electronic device to:
control a change in at least one of a driving mode and driving route of the vehicle, based on determining that the specified condition is satisfied.

15. A method for providing biometric information in a wearable device and an electronic device comprised in a vehicle, the method comprising:
obtaining, by the wearable device, first data used to calculate a user's biometric level by using at least one sensor, and transmitting the first data to the electronic device by using a first communication interface;
calculating, by the electronic device, the user's biometric level by using the first data, based on receiving the first data from the wearable device by using a second communication interface;
outputting, by the electronic device, the calculated user's biometric level by using at least one of a display and an audio interface;
outputting, by the electronic device, guide information related to a change in at least part of the biometric level by using at least one of the display and the audio interface, based on determining that a specified condition is satisfied, and transmitting the guide information to the wearable device by using the second communication interface;
obtaining, by the wearable device, second data related to properties of the guide information by using the at least one sensor, based on receiving the guide information from the electronic device by using the first communication interface, and transmitting the second data to the electronic device by using the first communication interface; and
generating, by the electronic device, feedback information about the guide information by using the second data, based on receiving the second data from the wearable device by using the second communication interface, and outputting the feedback information by using at least one of the display and the audio interface.

16. One or more non-transitory computer-readable storage media storing one or more computer programs including computer-executable instructions that, when executed by one or more processors of a wearable device individually or collectively, cause the wearable device to perform operations, the operations comprising:
obtaining first data used to calculate a user's biometric level by using at least one sensor of the wearable device;
transmitting the first data to an external electronic device;
receiving guide information related to a change in at least part of the biometric level from the external electronic device, based on the transmission of the first data;
obtaining second data related to properties of the guide information by using the at least one sensor, based on the reception of the guide information; and
transmitting the second data to the external electronic device.

17. The one or more non-transitory computer-readable storage media of claim 16, the operations further comprising transmitting third data comprising resting HR data recorded in an application program stored in the wearable device to the external electronic device, at a time of establishing communication with the external electronic device or at a time of initially transmitting the first data to the external electronic device based on a specified period.
